SEQUENCE LISTING (55 sequences)

SEQ ID NO: 1 - protein, 960 aa, Artificial Sequence
  CDKL5107 isoform polypeptide 1-960 (full- length)
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 2 - protein, 852 aa, Artificial Sequence
  CDKL5107 Variant ?853-960
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHP

SEQ ID NO: 3 - protein, 744 aa, Artificial Sequence
  CDKL5107 Variant ?745-960
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESS

SEQ ID NO: 4 - protein, 636 aa, Artificial Sequence
  CDKL5107 Variant ?637-960
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMA

SEQ ID NO: 5 - protein, 528 aa, Artificial Sequence
  CDKL5107 Variant ?529-960
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPT

SEQ ID NO: 6 - protein, 420 aa, Artificial Sequence
  CDKL5107 Variant ?421-960
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFN

SEQ ID NO: 7 - protein, 314 aa, Artificial Sequence
  CDKL5107 Variant ?315-960
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRK

SEQ ID NO: 8 - protein, 854 aa, Artificial Sequence
  CDKL5107 Variant ?315-420
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 9 - protein, 746 aa, Artificial Sequence
  CDKL5107 Variant ?315-528
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 10 - protein, 638 aa, Artificial Sequence
  CDKL5107 Variant ?315-636
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 11 - protein, 530 aa, Artificial Sequence
  CDKL5107 Variant ?315-744
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 12 - protein, 422 aa, Artificial Sequence
  CDKL5107 Variant ?315-852
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 13 - protein, 27 aa, Artificial Sequence
  TAT28 CPP
DAAQPARRARRTKLAAYGRKKRRQRRR

SEQ ID NO: 14 - protein, 27 aa, Artificial Sequence
  TAT?28 CPP
DAAQPARRARRTKLAAYARKAARQARA

SEQ ID NO: 15 - protein, 11 aa, Artificial Sequence
  TAT11 CPP
YGRKKRRQRRR

SEQ ID NO: 16 - protein, 11 aa, Artificial Sequence
  TAT?11 CPP
YARKAARQARA

SEQ ID NO: 17 - protein, 21 aa, Artificial Sequence
  Transportan CPP
AGYLLGKINLKALAALAKKIL

SEQ ID NO: 18 - protein, 16 aa, Artificial Sequence
  Antennapedia CPP
RQIKIWFQNRRMKWKK

SEQ ID NO: 19 - protein, 1064 aa, Artificial Sequence
  >MBip_Tk28p_107_3xFlagHis_cho-opt in pOptiVec
MKLSLVAAMLLLLSLVAAMLLLLSAARAGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 20 - protein, 1056 aa, Artificial Sequence
  >IgK_Tk28p_107_3xFlagHis_cho-opt in pOptiVec
METDTLLLWVLLLWVPGSTGGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 21 - protein, 1134 aa, Artificial Sequence
  >MBiP_Tk28p_115_3xFlagHis_cho-opt in pOptiVec
MKLSLVAAMLLLLSLVAAMLLLLSAARAGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPDGGCDGRRQRHHSGPQDRRFMLRTTEQQGEYFCCGDPKKPHTPCVPNRALHRPISSPAPYPVLQVRGTSMCPTLQVRGTDAFSCPTQQSGFSFFVRHVMREALIHRAQVNQAALLTYHENAALTGKGGGGSENLYFQGDYKDHDGDYKDHDIDYKDDDDKDGAPHHHHHH

SEQ ID NO: 22 - protein, 1126 aa, Artificial Sequence
  >IgK_Tk28p_115_3xFlagHis_cho-opt in pOptiVec
METDTLLLWVLLLWVPGSTGGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPDGGCDGRRQRHHSGPQDRRFMLRTTEQQGEYFCCGDPKKPHTPCVPNRALHRPISSPAPYPVLQVRGTSMCPTLQVRGTDAFSCPTQQSGFSFFVRHVMREALIHRAQVNQAALLTYHENAALTGKGGGGSENLYFQGDYKDHDGDYKDHDIDYKDDDDKDGAPHHHHHH

SEQ ID NO: 23 - protein, 1037 aa, Artificial Sequence
  >Tk28p_107_3xFlagHis_cho-opt in pOptiVec
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 24 - protein, 1037 aa, Artificial Sequence; identical to SEQ ID NO: 23
  >Tk28p_107_3xFlagHis_ecoli-opt in pEX-1

SEQ ID NO: 25 - protein, 929 aa, Artificial Sequence
  >?853-960 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPGGGGSENLYFQGDYKDHDGDYKDHDIDYKDDDDKDGAPHHHHHH

SEQ ID NO: 26 - protein, 821 aa, Artificial Sequence
  >?745-960 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSGGGGSENLYFQGDYKDHDGDYKDHDIDYKDDDDKDGAPHHHHHH

SEQ ID NO: 27 - protein, 713 aa, Artificial Sequence
  >?637-960 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAGGGGSENLYFQGDYKDHDGDYKDHDIDYKDDDDKDGAPHHHHHH

SEQ ID NO: 28 - protein, 605 aa, Artificial Sequence
  >?529-960 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTGGGGSENLYFQGDYKDHDGDYKDHDIDYKDDDDKDGAPHHHHHH

SEQ ID NO: 29 - protein, 497 aa, Artificial Sequence
  >?421-960 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNGGGGSENLYFQGDYKDHDGDYKDHDIDYKDDDDKDGAPHHHHHH

SEQ ID NO: 30 - protein, 391 aa, Artificial Sequence
  >?315-960 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKGGGGSENLYFQGDYKDHDGDYKDHDIDYKDDDDKDGAPHHHHHH

SEQ ID NO: 31 - protein, 931 aa, Artificial Sequence
  >?315-420 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 32 - protein, 823 aa, Artificial Sequence
  >?315-528 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 33 - protein, 715 aa, Artificial Sequence
  >?315-636 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 34 - protein, 607 aa, Artificial Sequence
  >?315-744 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 35 - protein, 499 aa, Artificial Sequence
  >?315-852 in pEX-1
MGDAAQPARRARRTKLAAYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 36 - protein, 1037 aa, Artificial Sequence
  >Tt28p_107_3xFlagHis_ecoli-opt in pEX-1
MGDAAQPARRARRTKLAAYGRKKRRQRRRGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 37 - protein, 316 aa, Artificial Sequence
  >Tk28p_eGFP_ecoli-opt_3xFlagHis in pEX-1
MGDAAQPARRARRTKLAAYARKAARQARAGGGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGGGSENLYFQGDYKDHDGDYKDHDIDYKDDDDKDGAPHHHHHH

SEQ ID NO: 38 - protein, 283 aa, Artificial Sequence
  >eGFP_3xFlagHis_ecoli-opt in pEX-1
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGGGSENLYFQGDYKDHDGDYKDHDIDYKDDDDKDGAPHHHHHH

SEQ ID NO: 39 - protein, 739 aa, Artificial Sequence
  >AMPH1-3xFlagHis in pEX-1 (ecoli-opt)
MADIKTGIFAKNVQKRLNRAQEKVLQKLGKADETKDEQFEEYVQNFKRQEAEGTRLQRELRGYLAAIKGMQEASMKLTESLHEVYEPDWYGREDVKMVGEKCDVLWEDFHQKLVDGSLLTLDTYLGQFPDIKNRIAKRSRKLVDYDSARHHLEALQSSKRKDESRISKAEEEFQKAQKVFEEFNVDLQEELPSLWSRRVGFYVNTFKNVSSLEAKFHKEIAVLCHKLYEVMTKLGDQHADKAFTIQGAPSDSGPLRIAKTPSPPEEPSPLPSPTASPNHTLAPASPAPARPRSPSQTRKGPPVPPLPKVTPTKELQQENIISFFEDNFVPEISVTTPSQNEVPEVKKEETLLDLDFDPFKPEVTPAGSAGVTHSPMSQTLPWDLWTTSTDLVQPASGGSFNGFTQPQDTSLFTMQTDQSMICNLAESEQAPPTEPKAEEPLAAVTPAVGLDLGMDTRAEEPVEEAVIIPGADADAAVGTLVSAAEGAPGEEAEAEKATVPAGEGVSLEEAKIGTETTEGAESAQPEAEELEATVPQEKVIPSVVIEPASNHEEEGENEITIGAEPKETTEDAAPPGPTSETPELATEQKPIQDPQPTPSAPAMGAADQLASAREASQELPPGFLYKVETLHDFEAANSDELTLQRGDVVLVVPSDSEADQDAGWLVGVKESDWLQYRDLATYKGLFPENFTRRLDENLYFQGGGGGSDYKDHDGDYKDHDIDYKDDDDKDGAPHHHHHH

SEQ ID NO: 40 - protein, 739 aa, Artificial Sequence; identical to SEQ ID NO: 39
  >AMPH1-3xFlagHis cho-opt in pOptiVec

SEQ ID NO: 41 - protein, 1048 aa, Artificial Sequence
  >MBip_Tatk11_107_3xFlagHis_cho-opt in pOptiVec
MKLSLVAAMLLLLSLVAAMLLLLSAARAGYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 42 - protein, 1040 aa, Artificial Sequence
  >IgK_Tatk11_107_3xFlagHis_cho-opt in pOptiVec
METDTLLLWVLLLWVPGSTGGYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 43 - protein, 1021 aa, Artificial Sequence
  >Tatk11_107_3xFlagHis_cho-opt in pOptiVec (leaderless)
MGYARKAARQARAGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 44 - protein, 1021 aa, Artificial Sequence; identical to SEQ ID NO: 43
  >Tatk11_107_3xFlagHis_ecoli-opt in pEX-1

SEQ ID NO: 45 - protein, 1021 aa, Artificial Sequence
  >Tat11_107_3xFlagHis_ecoli-opt in pEX-1
MGYGRKKRRQRRRGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 46 - protein, 1021 aa, Artificial Sequence; identical to SEQ ID NO: 45
  >Tat11_107_3xFlagHis_cho-opt in pOptiVec (leaderless)

SEQ ID NO: 47 - protein, 1030 aa, Artificial Sequence
  CDKL5115 isoform polypeptide 1-1030 (full-length)
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPDGGCDGRRQRHHSGPQDRRFMLRTTEQQGEYFCCGDPKKPHTPCVPNRALHRPISSPAPYPVLQVRGTSMCPTLQVRGTDAFSCPTQQSGFSFFVRHVMREALIHRAQVNQAALLTYHENAALTGK

SEQ ID NO: 48 - protein, 28 aa, Artificial Sequence
  MBiP
MKLSLVAAMLLLLSLVAAMLLLLSAARA

SEQ ID NO: 49 - protein, 20 aa, Artificial Sequence
  Murine Ig?
METDTLLLWVLLLWVPGSTG

SEQ ID NO: 50 - protein, 12 aa, Artificial Sequence
  P97
DSSHAFTLDELR

SEQ ID NO: 51 - protein, 25 aa, Artificial Sequence
  MBiP2
MKLSLVAAMLLLLWVALLLLSAARA

SEQ ID NO: 52 - protein, 26 aa, Artificial Sequence
  MBiP3
MKLSLVAAMLLLLSLVALLLLSAARA

SEQ ID NO: 53 - protein, 26 aa, Artificial Sequence
  MBiP4
MKLSLVAAMLLLLALVALLLLSAARA

SEQ ID NO: 54 - protein, 1026 aa, Artificial Sequence
  >ANTP_107_3xFlagHis_cho-opt in pOptiVec
MGRQIKIWFQNRRMKWKKGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

SEQ ID NO: 55 - protein, 1031 aa, Artificial Sequence
  >TRANSP_107_3xFlagHis_cho-opt in pOptiVec
MGAGYLLGKINLKALAALAKKILGGGGSKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETALGGGGSENLYFQGDYKDHDGDYKDHDIDYKDDDDKDGAPHHHHHH

(19) United States
(12) Patent Application Publication
(10) Pub. No.: US 2020/0299654 A1
(43) Pub. Date: Sep. 24, 2020

(54) CDKL5 EXPRESSION VARIANTS AND CDKL5 FUSION PROTEINS

(71) Applicant: Amicus Therapeutics, Inc., Cranbury, NJ (US)

(72) Inventor: Sean Clark, Montgomery Township, NJ (US)

(73) Assignee: Amicus Therapeutics, Inc., Cranbury, NJ (US)

(21) Appl. No.: 16/768,511

(22) PCT Filed: Nov. 30, 2018
(86) PCT No.: PCT/US2018/063294

§ 371 Date: May 29, 2020

Related U.S. Application Data
(60) Provisional application No. 62/592,944, filed on Nov. 30, 2017.
(60) Provisional application No. 62/592,936, filed on Nov. 30, 2017.

(51) Int. Cl. C12N 9/12; A61K 9/00

(52) U.S. Cl. CPC C12N 9/12; C12Y 207/11022; A61K 38/00; C07K 2319/02; A61K 9/0019

(57) ABSTRACT

Novel CDKL5 enzyme variants are provided, as well as fusion proteins comprising full-length CDKL5 polypeptides or CDKL5 variants. Such fusion proteins can include cell-penetrating polypeptides and optionally comprising a leader signal polypeptide and/or tags. Also provided are methods of producing such CDKL5 variants and fusion proteins, as well as pharmaceutical compositions, methods of treatment, and uses of such recombinant proteins.

### TECHNICAL FIELD

The present invention generally relates to the treatment of kinase deficiency disorders, particularly novel recombinant proteins for the treatment of disorders involving deficiency of CDKL5.

### BACKGROUND

CDKL5 is a serine/threonine kinase and was previously known as STK9. Mutations in this gene have recently been associated with a number of neurological disorders such as mental retardation, loss of communication and motor skills, infantile spasms and seizures, atypical Rett Syndrome, and X-linked West Syndromes. Mutations or deletions of the X-linked gene cyclin-dependent kinase-like 5 (CDKL5 ) have been shown to cause an epileptic encephalopathy with early-onset severe neurological impairment and intractable seizures.

Currently, the oldest known people described in medical literature with CDKL5 deficiency have reached an age of 41 years old. Many others are in their twenties and teens, but because the disease has only been identified in the last 15 years, the majority of newly diagnosed are toddlers or infants. Individuals diagnosed with CDKL5 deficiency disorder generally suffer delays in neurological development and are at a high risk for seizures, with a median onset age of 6 weeks. One study of 111 participants found that 85.6% of individuals had epilepsy with a daily occurrence of seizures, and a mean of 6 seizures per day.

Current treatments range from seizure medications, ketogenic diets, vagal nerve stimulation, and surgery. Commonly administered anti-epileptic medications include clobazam, valproic acid, and topiramate, and in many cases two or more medication regiments are used at the same time. Individuals seemed to have a “honeymoon period” in which they are seizure free for a period of time after starting a new type of medication, but ultimately there is a recurrence of seizures. The duration of observed honeymoon ranges from 2 months to 7 years, with a median of 6 months. For example, the study found that 16 of the 111 participants were currently seizure free, and one individual had never developed seizures.

The exact mechanisms for pathogenic manifestations remain unclear. Some experimental data suggest that certain non-sense mutations in the C-terminus cause the protein to be constitutively localized to the nucleus, while other missense mutations are highly represented in the cytoplasm. Nuclear localization signals and nuclear export signals have both been identified in the C-terminus of the protein.

Some mutant enzyme variants result in partial or total loss of phosphorylation function, while other mutations and truncations result in an increase in phosphorylation capacity, suggesting that both loss and gain of function may be pathogenic. Interactions and pathogenic effects arising from enzymatic activity loss/gain of function and enzyme nuclear localization versus residence in the cytoplasm remain unclear. An analysis of patients with a wide range of CDKL5 mutations and presenting clinical symptoms suggests that mutations causing clinical symptoms are more likely to be found either in the C-terminus or the kinase activity domain, suggesting that both the kinase activity and protein translocation capacity of CDKL5 could affect the clinical manifestation of symptoms.

### SUMMARY

Accordingly, various aspects of the invention pertain to new CDKL5 variants and CDKL5 fusion proteins, which can be used to treat CDKL5-mediated neurological disorders such as a CDKL5 deficiency or an atypical Rett syndrome caused by a CDKL5 mutation or deficiency. Other aspects of the invention pertain to methods of producing such CDKL5 variants and fusion proteins, as well as pharmaceutical compositions, methods of treatment, and uses of such recombinant proteins.

One aspect of the present invention is related to a CDKL5 polypeptide as described herein. In one or more embodiments, the CDKL5 polypeptide comprises a sequence having at least 98% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12. In one or more embodiments, the CDKL5 polypeptide comprises a sequence having at least 99% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12. In one or more embodiments, the CDKL5 polypeptide comprises a sequence having 100% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12.

Another aspect of the present invention is related to a CDKL5 polypeptide lacking a nuclear export signal (NES). In one or more embodiments, the CDKL5 polypeptide contains a nuclear localization signal (NLS).

Another aspect of the present invention is related to a CDKL5 polypeptide lacking a nuclear localization signal (NLS) and containing a nuclear export signal (NES).

Another aspect of the present invention is related to a fusion protein comprising a CDKL5 polypeptide as described herein and a cell-penetrating polypeptide. In one or more embodiments, the cell-penetrating polypeptide has at least 90% sequence identity to SEQ ID NO: 13, SEQ ID NO: 14, SEQ ID NO: 15, SEQ ID NO: 16, SEQ ID NO: 17, SEQ ID NO: 18 or SEQ ID NO: 50. In one or more embodiments, the cell-penetrating polypeptide has at least 95% sequence identity to SEQ ID NO: 13, SEQ ID NO: 14, SEQ ID NO: 15, SEQ ID NO: 16, SEQ ID NO: 17, SEQ ID NO: 18 or SEQ ID NO: 50. In one or more embodiments, the cell-penetrating polypeptide has 100% sequence identity to SEQ ID NO: 13, SEQ ID NO: 14, SEQ ID NO: 15, SEQ ID NO: 16, SEQ ID NO: 17, SEQ ID NO: 18 or SEQ ID NO: 50. In one or more embodiments, the cell-penetrating polypeptide has at least 90% sequence identity to SEQ ID NO: 13, SEQ ID NO: 14, SEQ ID NO: 15, SEQ ID NO: 16, SEQ ID NO: 17 or SEQ ID NO: 18. In one or more embodiments, the cell-penetrating polypeptide has at least 95% sequence identity to SEQ ID NO: 13, SEQ ID NO: 14, SEQ ID NO: 15, SEQ ID NO: 16, SEQ ID NO: 17 or SEQ ID NO: 18. In one or more embodiments, the cell-penetrating polypeptide has 100% sequence identity to SEQ ID NO: 13, SEQ ID NO: 14, SEQ ID NO: 15, SEQ ID NO: 16, SEQ ID NO: 17 or SEQ ID NO: 18. In one or more embodiments, the cell-penetrating polypeptide has at least 90% sequence identity to SEQ ID NO: 13, SEQ ID NO: 14, SEQ ID NO: 15, SEQ ID NO: 17 or SEQ ID NO: 18. In one or more embodiments, the cell-penetrating polypeptide has at least 95% sequence identity to SEQ ID NO: 13, SEQ ID NO: 14, SEQ ID NO: 15, SEQ ID NO: 17 or SEQ ID NO: 18. In one or more embodiments, the cell-penetrating polypeptide has 100% sequence identity to SEQ ID NO: 13, SEQ ID NO: 14, SEQ ID NO: 15, SEQ ID NO: 17 or SEQ ID NO: 18. In various embodiments, the CDKL5 polypeptide is a full-length CDKL5 polypeptide (e.g. as shown in SEQ ID NO. 1 or SEQ ID NO: 47). In other embodiments, the CDKL5 polypeptide is a variant as described herein (e.g. as shown in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12).

Another aspect of the present invention is related to a pharmaceutical formulation comprising a CDKL5 polypeptide as described herein or a fusion protein as described herein, and a pharmaceutically acceptable carrier.

Another aspect of the present invention is related to a method of treating a CDKL5-mediated neurological disorder, the method comprising administering a formulation comprising a CDKL5 polypeptide as described herein or a fusion protein as described herein; and a pharmaceutically acceptable carrier. In one or more embodiments, the formulation is administered intrathecally. In one or more embodiments, the formulation is administered intravenously. In one or more embodiments, the formulation is administered intracisternally. In one or more embodiments, the formulation is administered intracerebroventrically. In one or more embodiments, the formulation is administered intraparenchymally. In one or more embodiments, the CDKL5-mediated neurological disorder is one or more of a CDKL5 deficiency or an atypical Rett syndrome caused by a CDKL5 mutation or deficiency.

Another aspect of the present invention is related to a method of producing a CDKL5 polypeptide as described herein or a fusion protein as described herein. In one or more embodiments, the method comprises expressing the CDKL5 polypeptide or the fusion protein; and purifying the CDKL5 polypeptide or the fusion protein. In one or more embodiments, the CDKL5 polypeptide or the fusion protein is expressed in Chinese hamster ovary (CHO) cells, HeLa cells, human embryonic kidney (HEK) cells or Escherichia coli cells.

Another aspect of the present invention is related to a polynucleotide encoding a CDKL5 polypeptide as described herein or a fusion protein as described herein. Another aspect of the present invention is related to a vector comprising such a polynucleotide.

### BRIEF DESCRIPTION OF THE DRAWINGS

FIG. 1A shows a polypeptide map of CDKL5₁₀₇. The map identifies important features of the polypeptide, including the ATP binding site, kinase domain and kinase active site, two nuclear localization signals, and a nuclear export signal.

FIGS. 1B and 1C show a graphic depicting the synthesized CDKL5 construct variants (1B) and a legend describes the length of the polypeptides, along with the relevant amino acid deletion information to describe how the constructs were synthesized (1C).

FIGS. 2A-2AD show exemplary plasmids for expressing various fusion proteins in cells such as CHO cells or E. Coli cells.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Various aspects of the invention pertain to new CDKL5 variants and CDKL5 fusion proteins. Other aspects of the invention pertain to methods of producing such CDKL5 variants and fusion proteins, as well as pharmaceutical compositions, methods of treatment, and uses of such recombinant proteins.

Without wishing to be bound by any particular theory, it is believed that shorter CDKL5 variants that retain functional activity can provide benefits over the full-length CDKL5 polypeptide, particularly when incorporated into a fusion protein comprising the CDKL5 polypeptide. In one or more embodiments, such benefits can include improved secretion from host cells during protein production, improved solubility, enhanced ability to cross the blood-brain barrier (BBB), and/or enhanced ability to penetrate target cells.

### Definitions

As used herein, a “CDKL5-mediated neurological disorder” refers to any disease or disorder that can be treated by expression or overexpression of the CDKL5 protein.

As used herein, “CDKL5 deficiency” refers to any deficiency in the biological function of the protein. The deficiency can result from any DNA mutation in the DNA coding for the protein or a DNA related regulatory region or any change in the function of the protein due to any changes in epigenetic DNA modification, including but not limited to DNA methylation or histone modification, any change in the secondary, tertiary, or quaternary structure of the CDKL5 protein, or any change in the ability of the CDKL5 protein to carry out its biological function as compared to a wild-type or normal subject. The deficiency can also include a lack of CDKL5 protein, such as a null mutation or underexpression of a fully functioning protein.

As used herein, “an atypical Rett syndrome caused by a CDKL5 mutation or deficiency” refers to an atypical form of Rett syndrome with similar clinical signs to Rett syndrome but is caused by a CDKL5 mutation or deficiency.

Symptoms or markers of a CDKL5 deficiency, Rett syndrome, or an atypical Rett syndrome include but are not limited to seizures, cognitive disability, hypotonia, as well as autonomic, sleep, and gastrointestinal disturbances.

As used herein, the term “carrier” is intended to refer to a diluent, adjuvant, excipient, or vehicle with which a compound is administered. Suitable pharmaceutical carriers are known in the art and, in at least one embodiment, are described in “Remington's Pharmaceutical Sciences” by E. W. Martin, 18th Edition, or other editions.

As used herein, the term “enzyme replacement therapy” or “ERT” is intended to refer to the introduction of an exogenous, purified enzyme into an individual having a deficiency in such enzyme. The administered protein can be obtained from natural sources or by recombinant expression. The term also refers to the introduction of a purified enzyme in an individual otherwise requiring or benefiting from administration of a purified enzyme. In at least one embodiment, such an individual suffers from enzyme insufficiency. The introduced enzyme may be a purified, recombinant enzyme produced in vitro, or a protein purified from isolated tissue or fluid, such as, for example, placenta or animal milk, or from plants.

As used herein, the terms “subject” or “patient” are intended to refer to a human or non-human animal In at least one embodiment, the subject is a mammal. In at least one embodiment, the subject is a human.

As used herein, the “therapeutically effective dose” and “effective amount” are intended to refer to an amount of recombinant proteins (e.g. CDKL5 variants or fusion proteins) which is sufficient to result in a therapeutic response in a subject. A therapeutic response may be any response that a user (for example, a clinician) will recognize as an effective response to the therapy, including any surrogate clinical markers or symptoms described herein and known in the art. Thus, in at least one embodiment, a therapeutic response can be an amelioration or inhibition of one or more symptoms or markers of a CDKL5 deficiency, Rett syndrome, or an atypical Rett syndrome such as those known in the art.

### Function of CDKL5 Proteins

The human CDKL5 gene is composed of 24 exons, of which the first three (exons 1, 1a and 1b) are untranslated.

The originally discovered human CDKL5 variant was 1030 amino acids with a molecular mass of 115 kDa (CDKL5₁₁₅). Another prominent variant, CDKL5₁₀₇, contains an altered C-terminal region because alternative splicing combines different exons than in the CDKL5₁₁₅ variant. CDKL5₁₀₇ (107 kDa) is shorter because it harbors an alternate version of exon 19 and does not contain exons 20-21 that are present in the CDKL5₁₁₅ variant. The hCDKL5₁₀₇ mRNA has been found to be 37-fold more abundant in human brain than the hCDKL5₁₁₅ transcript, and murine CDKL5₁₀₇ has been found to be 160-fold more abundant than the murine CDKL5₁₀₅ variant in murine brain. Both the human and murine CDKL5₁₀₇ isoforms have demonstrated a longer half-life and resistance to degradation as compared to the human CDKL5₁₁₅ variant.

CDKL5 knockout mouse models have been generated using the Lox-Cre recombination system and these mice present symptoms of autistic-like deficits in social interactions, impairment of motor control, and loss of fear memory (Wang et al., Proc Natl Acad Sci U.S.A, 109(52), 21516-21521). For example, knockout CDKL5 mice have symptoms of reduced motor coordination and demonstrate impaired memory and fear responses when repeatedly exposed to stimuli. These changes have led scientists to hypothesize that loss of CDKL5 kinase activity leads to impaired neuronal network development. Previous data have suggested that CDKL5 phosphorylates methyl-CpG binding protein 2 (MeCP2), and independent loss-of-function mutations in MeCP2 lead to the Rett syndrome phenotype. Other substrates of CDKL5 include Netrin G1 ligand (NGL-1), Shootinl (SHTN1), Mindbomb 1 (MIB1), DNA (cytosine-5)-methyltransferase 1 (DNMT1), Amphiphysin 1 (AMPH1), end-binding protein EB2, microtubule associated protein 1S (MAP1S) and histone deacetylase 4 (HDAC4). Although the exact role of CDKL5 has yet to be identified, these data suggest that CDKL5 plays a role in phosphorylation of downstream targets that are critical for correct neuronal development, including MeCP2. In humans, mutations in CDKL5 are associated with a phenotype that overlaps with Rett syndrome, and additionally presents with early-onset seizures. While CDKL5 KO mice did not exhibit any early-onset seizure symptoms, they did exhibit motor defects, decreased sociability, and impaired learning and memory (Chen et al. CDKL5 , a protein associated with Rett Syndrome, regulates neuronal morphogenesis via Racl signaling, J Neurosci 30: 12777-12786)

Two CDKL5 isoforms are found in rat, one labeled CDKL5a and the other CDKL5b. (Chen et al.). In general, there is a high level of sequence conservation in CDKL5 genes across human, rat, and mouse species except for the last 100-150 amino acids near the C-terminus. Western blot data show that both variants are present during rat development yet adults appear to predominately express a single variant. Furthermore, CDKL5 is present in identifiable quantities in brain, liver, and lung.

CDKL5 functions in the nucleus but it is also found in the dendrites of cultured neurons, suggesting a possible alternate cytoplasmic role. Down regulation of CDKL5 expression by RNAi (RNA Interference) in cultured cortical neurons inhibited neurite growth and dendritic arborization (branching), where over expression of CDKL5 had opposite effects (Chen et al.). In order to characterize both the nuclear and cytoplasmic effect of CDKL5, a variant of CDKL5a with a nuclear export sequence (NES) was expressed in the cultured cortical neuron RNAi model. This NES-CDKL5a variant was resistant to the RNAi used to silence the wild-type gene expression, and therefore was used to model CDKL5a when expressed solely in the cytoplasm. After using the GFP tag to confirm that this CDKL5 variant was exclusively present in the cytoplasm, an increase in both the length of neurites and number of neurite branches was seen. The ability of NES-GFP-CDKL5a to partially rescue the disease phenotype observed when RNAi was used to knockdown the endogenous CDKL5 expression suggests that the expression of CDKL5 in cytoplasm in an important factor in the development and growth of neurites.

Human mutations in CDKL5 are associated with a phenotype similar to Rett syndrome, and individuals with CDKL5 mutations also present with early-onset seizures. This onset of seizures differs from the classical Rett syndrome phenotype in which there is an early normal period of development before the onset of Rett symptoms. Patients with classical Rett syndrome (RTT) appear to develop normally until 6-18 months of age, and then they begin to present neurological symptoms including loss of speech and movement. Autopsies of RTT brains show smaller and more densely packed neurons with shorter dendrites in the motor and frontal cortex, suggesting that neuronal development is impaired. The majority of Classical RTT cases are due to mutations in the MECP2 gene, which is an X-linked gene encoding a nuclear protein that selectively binds to CpG dinucleotides in the mammalian genome and regulates transcription through the recruitment of complexes. Although poorly understood, it is generally thought that the dysregulation of gene expression caused by mutations in MECP2 is the underlying cause of Rett Syndrome. Approximately 20% of Classic Rett syndrome cases and 60-80% of other Rett syndrome variants carry no mutations in MECP2, suggesting an alternate genetic cause for pathogenesis. Recently, some CDKL5 mutations have been identified in patients with certain variants of RTT and other severe encephalopathies, and CDKL5 has been shown to interact with MeCP2 both in vivo and in vitro. Beyond MeCP2, CDKL5 has been shown to interact with and phosphorylate a number of downstream targets, including NGL-1. When phosphorylated, NGL-1 interacts with PSD95 and is critical for the correct genesis and development of dendritic spines and synapse formation (Ricciardi S, et al. “CDKL5 ensures excitatory synapse stability by reinforcing NGL-1-PSD95 interaction in the postsynaptic compartment and is impaired in patient iPSC-derived neurons.” Nat Cell Biol 14(9):911-923).

CDKL5 has also been shown to phosphorylate the protein DNA methyltransferase 1 (DNMT1) (Kameshita I, et al. “Cyclin-dependent kinase-like 5 binds and phosphorylates DNA methyltransferase 1.” Biochem Biophys Res Commun 377:1162-1167). This phosphorylation leads to activation of DNMT1, which is a maintenance-type methylation protein that preferentially methylates hemimethylated DNA. This process is useful for maintenance of DNA methylation patterns during DNA replication, so that newly synthesized daughter DNA strands are able to maintain the methylation pattern of the parent strand it replaced. As methylation of DNA is generally thought to be an epigenetic mechanism to silence gene expression, this maintenance function of DNMT1 is crucial in preserving gene expression patterns across cell generations.

Current models suggest that the CDKL5 kinase domain phosphorylates GSK-3β, and that phosphorylation of GSK-3β leads to its inactivation. Individuals who are deficient in CDKL5 activity therefore seem to exhibit increased GSK-3β activity. Previous studies have shown that GSK-3β modulates hippocampal neurogenesis, and that an increased activity of GSK-3β severely impairs dendritic morphology of newborn hippocampal neurons. Furthermore, GSK-3β seems to act as a negative regulator of key developmental events such as neuron survival and maturation. A study conducted using CDKL5 KO mice demonstrated that treatment with a GSK-3β inhibitor can almost fully rescue hippocampal development and behavioral deficits in mice deficient in CDKL5 activity (Fuchs et al. “Inhibition of GSK3β Rescues Hippocampal Development and Learning in a Mouse Model of CDKL5 Disorder.” Neurobiology of Disease 82: 298-310.). This developmental rescue also seemed to persist beyond treatment.

### CDKL5₁₀₇ Polypeptide Constructs

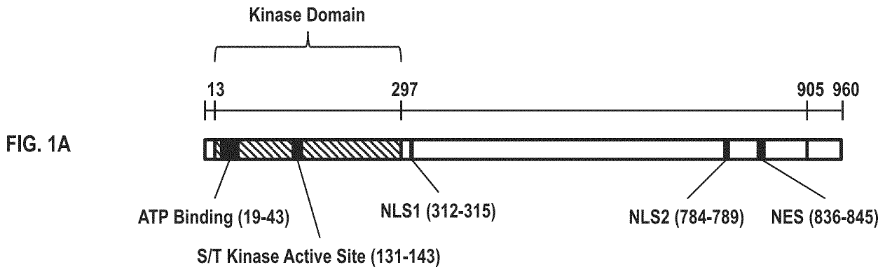

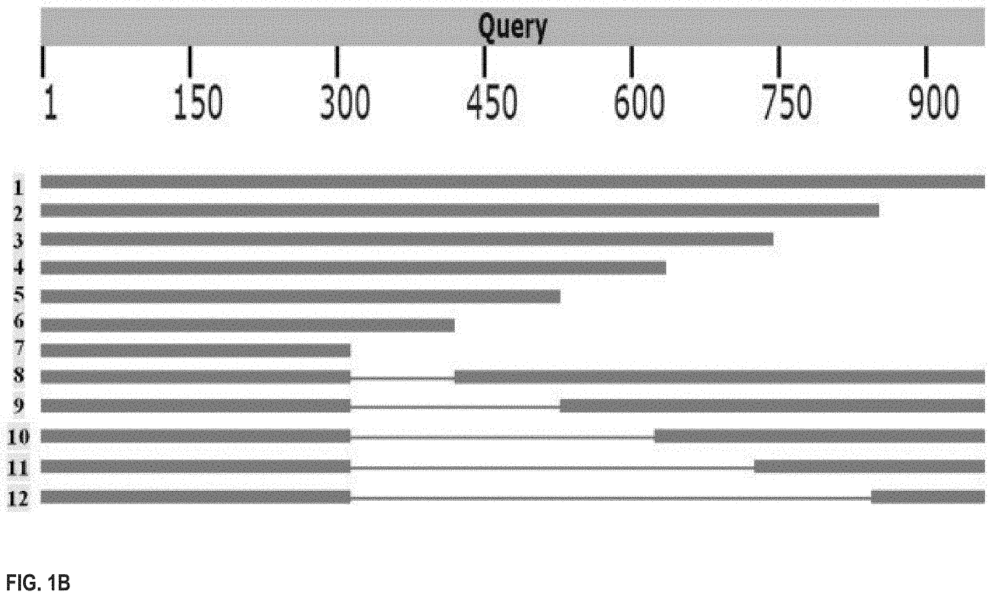

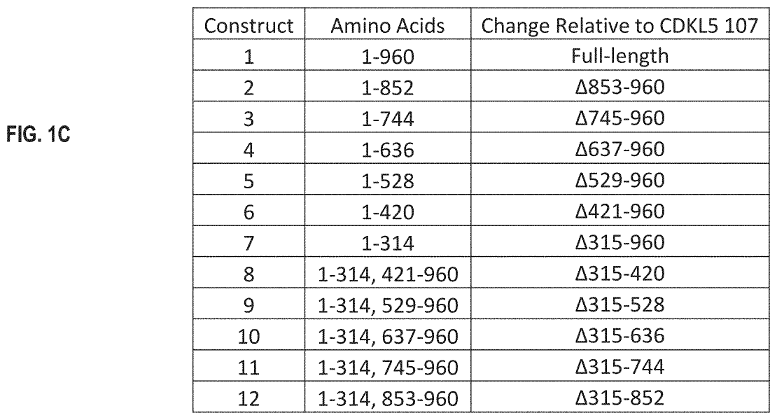

FIG. 1A displays a polypeptide map of CDKL5₁₀₇. The amino acid sequence of the wild-type full-length human CDKL5₁₀₇ isoform is provided in SEQ ID NO: 1. The CDKL5₁₀₇ protein consists of 960 amino acids, and the kinase domain is contained in the first ˜300 amino acids. Residue 42 of 960 is a key lysine residue located within the kinase domain that participates in ATP binding during a phosphorylation reaction, and mutation of this residue generally leads to loss of kinase activity (“Kinase dead”). Additionally, two nuclear localization signals are present spanning residues 312-315 (NLS1) and 784-789 (NLS2), and a nuclear export signal (NES) is present spanning residues 836-845 Amino acids at the C-terminus spanning from residue 905 to 960 are unique to CDKL5₁₀₇ and are not present in CDKL5₁₁₅. Amino acid residues 1-904 are identical between CDKL5₁₁₅ and CDKL5₁₀₇. The amino acid sequence of the wild-type full-length human CDKL5₁₁₅ isoform is provided in SEQ ID NO: 47.

Various embodiments of the present invention provide novel CDKL5 variants. FIGS. 1B and 1C show the polypeptides of the full-length human CDKL5₁₀₇ isoform (Construct 1) and novel CDKL5 constructs (designated as Constructs 2-12). These CDKL5 constructs generally fall into two categories: those missing some number of amino acids at the C-terminus (Constructs 2-7) and those missing some number of amino acids in the middle of the polypeptide chain (Constructs 8-12). Moreover, in those constructs wherein CDKL5 is fused C-terminally to additional N-terminal amino acid sequences, the initial methionine of CDKL5 is removed. In these constructs, the CDKL5 polypeptide begins with the second amino acid, lysine. Construct 1 contains all 960 amino acids of the full-length human CDKL5₁₀₇ isoform. Construct 2, which contains the first 851 amino acids of the entire 960 amino acid chain, represents a shortened CDKL5 polypeptide in which the tail sequence that differs between CDKL5₁₀₇ and CDKL5₁₁₅ is removed but the kinase domain, nuclear localization signals (NLS1 and NLS2), and nuclear export signal (NES) remain intact. Construct 3 is shortened further, in which the nuclear localization signal (NLS2) and the nuclear export signal (NES) are additionally removed. Constructs 4-7 are shortened even further, as shown in FIGS. 1B and 1C. Constructs 2-7 all contain the active kinase domain, while Constructs 3-7 do not contain the NLS2 or NES sequences. Construct 7 is further shortened up to the NLS1 sequence. The remaining constructs (Constructs 8-12) all have deletions in the middle portion of the polypeptide chain while retaining the C-terminal amino acids unique to CDKL5₁₀₇. Of these constructs, Construct 12 is missing the NES and NLS2 sequences. The amino acid sequences of Constructs 1-12 are provided in SEQ ID NOS: 1-12, respectively.

In one or more embodiments, the CDKL5 polypeptide has at least 98%, at least 98.5%, at least 99% or at least 99.5% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12. The CDKL5 polypeptide may contain deletions, substitutions and/or insertions relative to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12, such as having 1, 2, 3, 4, 5, 6, 7, 8, 9, 10, 11, 12, 13, 14, 15 or more deletions, substitutions and/or insertions to the amino acid sequence described by SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12.

In one or more embodiments, the CDKL5 polypeptide has at least 98%, at least 98.5%, at least 99% or at least 99.5% sequence identity to SEQ ID NO: 1 or SEQ ID NO: 47. The CDKL5 polypeptide may contain deletions, substitutions and/or insertions relative to SEQ ID NO: 1 or SEQ ID NO: 47, such as having 1, 2, 3, 4, 5, 6, 7, 8, 9, 10, 11, 12, 13, 14, 15 or more deletions, substitutions and/or insertions to the amino acid sequence described by SEQ ID NO: 1 or SEQ ID NO: 47.

Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 98%, 98.5%, 99% or 99.5% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

One skilled in the art can readily derive a polynucleotide sequence encoding a particular polypeptide sequence. Such polynucleotide sequence can be codon optimized for expression in the target cell using commercially available products, such as using the OptimumGene™ codon optimization tool (GenScript, Piscatway, N.J.).

### Cell-Penetrating Peptides (CPPs)

A variety of viral and cellular proteins possess basic polypeptide sequences that mediate translocation across cellular membranes. The capacity to translocate across cellular membranes has become an important tool for the delivery of high molecular weight polypeptides across membranes. The phrase “protein transduction domain” (PTD) and “cell-penetrating peptides” (CPPs) are usually used to refer to short peptides (<30 amino acids) that can traverse the plasma membrane of many, if not all, mammalian cells. After studies to identify the specific properties of the domain that allow them to collectively cross the plasma membrane, researchers have observed that these domains contain a large number of basic amino acid residues such as lysine and arginine. Thus, cell-penetrating peptides fall into two classes: the first consisting of amphipathic helical peptides that contain lysine residues which contribute a positive charge, while the second class includes arginine-rich peptides. These peptides could have therapeutic potential if used in combination with other proteins that are difficult to deliver to intracellular targets. The most frequent experimental uses of PTDs are TAT, Antennapedia (Antp), and other poly-arginine peptides.

Thus far, TAT has been the best characterized of the PTDs, and has been used to successfully deliver small cargoes, such as short peptides and oligonucleotides, to intercellular targets. HIV-TAT (HIV Transactivator of Transcription) is an 86-amino acid protein involved in the replication of human immunodeficiency virus type 1 (HIV-1), and many studies have shown that TAT is able to translocate through the plasma membrane and reach the nucleus in order to activate transcription of the viral genome. Studies have also shown that TAT retains its penetration properties when coupled to several different proteins. In an effort to understand which areas of the TAT protein are critical to the translocation property, experiments have been conducted in which different length peptide fragments of TAT are synthesized and their penetration capabilities are assessed. (Lebleu et al. “A Truncated HIV-1 TAT Protein Basic Domain Rapidly Translocates through the Plasma Membrane and Accumulates in the Cell Nucleus.” J. Biol. Chem. 1997, 272:16010-16017). A region of basic amino acids has been identified as the aspect of TAT that retains this penetration property, and experiments in which a TAT protein without this basic amino acid cluster is unable to penetrate the cellular plasma membrane. In some instances, the shorter sequence cell-penetrating peptide has been modified to prevent cleavage during secretion by endoprotease enzymes such as furin. These modifications change the shortened cell-penetrating TAT amino acid sequence from YGRKKRRQRRR to YARKAARQARA, and this short peptide is referred to as TATκ.

The exact mechanism in which TAT is able to translocate across the plasma membrane remains uncertain. Recent work has explored the possibility that a special type of endocytosis is involved with TAT uptake, and a few cell lines have been identified that appear resistant to TAT penetration. The specific cargo to be delivered by TAT may also play a role in the efficacy of delivery. Previous research data have suggested that a TAT fusion protein has better cellular uptake when it is prepared in denaturing conditions, because correctly folded protein cargo likely requires much more energy (delta-G) to cross the plasma membrane due to structural constraints.

The capacity of the intracellular protein chaperones to refold the TAT cargo likely varies based on the identity and size of the protein cargo to be re-folded. In some instances, TAT-fusion proteins precipitate when placed in an aqueous environment and therefore cannot be prepared in a denatured manner nor remain stable for very long in native conformations. The design of the TAT-fusion protein must also be tailored to the specific cargo to be delivered. If the cargo protein is tightly associated at the N-terminus and the TAT domain is also found at the N-terminus, the TAT translocation domain may be buried in the cargo protein and transduction may be poor.

Numerous TAT-cargo variants have been successfully delivered into a variety of cell types, including primary culture cells, transformed cells, and cells present in mouse tissue. In culture, the TAT-fusion proteins generally diffuse easily into and out of cells, leading to a very rapid establishment of uniform concentration.

Many pharmaceutical agents such as enzymes, antibodies, other proteins, or even drug-loaded carrier particles need to be delivered intracellularly to exert their therapeutic action inside the cytoplasm, nucleus, or other specific organelles. Thus, the delivery of these different types of large molecules represents a significant challenge in the development of biologics. Current data suggest that TAT is able to cross the plasma membrane through more than one mechanism.

A TAT transduction domain has also been fused to the enzyme superoxide dismutase (SOD). (Torchilin, “Intracellular delivery of protein and peptide therapeutics.” Protein Therapeutics. 2008. 5(2-3):e95-e103). This fusion protein was used to demonstrate that it could translocate across cell membranes in order to deliver the SOD enzyme to the intracellular environment, and thus here the fusion protein has therapeutic potential in treating enzyme deficiency disorders that lead to higher accumulation of reactive oxygen species and oxidative stress on a host cell.

TAT fusion proteins have also been shown to transduce across the blood brain barrier. A TAT domain fused to the neuroprotectant protein Bcl-xL was able to penetrate cells rapidly in culture, and when administered to mice suffering from cerebral ischemia, the fusion protein transduced brain cells within 1-2 hours. After transduction, the cerebral infarct was reduced in size in a dose-dependent manner (Cao, G. et al., “In Vivo Delivery of a Bcl-xL Fusion Protein Containing the TAT Protein Transduction Domain Protects against Ischemic Brain Injury and Neuronal Apoptosis.” J. Neurosci. 22, 5423, 2002.)

In various embodiments, the CDKL5 variants described herein are operably linked to a CPP such as TAT, modified TAT (TATκ), Transportan, Antennapedia or P97. As used herein, TAT can refer to the original TAT peptide having 11 amino acids (designated TAT11) or can refer to a TAT peptide having an additional 16 N-terminal amino acids (designated as TAT28) that are derived from the polylinker of the plasmid used for cloning. Similarly, TATκ can refer to a modified version of TAT11 (designated TATκ11) or a modified version of TAT28 (designated TATκ28). The amino acid sequences of the CPPs TAT28, TATκ28, TAT11, TATκ11, Transportan, Antennapedia and P97 are provided in SEQ ID NO: 13, SEQ ID NO: 14, SEQ ID NO: 15, SEQ ID NO: 16, SEQ ID NO: 17, SEQ ID NO: 18 and SEQ ID NO: 50, respectively.

In some embodiments, the CPP has at least 90% sequence identity to SEQ ID NO: 13, SEQ ID NO: 14, SEQ ID NO: 15, SEQ ID NO: 16, SEQ ID NO: 17, SEQ ID NO: 18 or SEQ ID NO: 50. In some embodiments, the CPP has at least 95% sequence identity to SEQ ID NO: 13, SEQ ID NO: 14, SEQ ID NO: 15, SEQ ID NO: 16, SEQ ID NO: 17, SEQ ID NO: 18 or SEQ ID NO: 50. In some embodiments, the CPP has 100% sequence identity to SEQ ID NO: 13, SEQ ID NO: 14, SEQ ID NO: 15, SEQ ID NO: 16, SEQ ID NO: 17, SEQ ID NO: 18 or SEQ ID NO: 50. In some embodiments, the CPP has at least 90% sequence identity to SEQ ID NO: 13, SEQ ID NO: 14, SEQ ID NO: 15, SEQ ID NO: 16, SEQ ID NO: 17 or SEQ ID NO: 18. In some embodiments, the CPP has at least 95% sequence identity to SEQ ID NO: 13, SEQ ID NO: 14, SEQ ID NO: 15, SEQ ID NO: 16, SEQ ID NO: 17 or SEQ ID NO: 18. In some embodiments, the CPP has 100% sequence identity to SEQ ID NO: 13, SEQ ID NO: 14, SEQ ID NO: 15, SEQ ID NO: 16, SEQ ID NO: 17 or SEQ ID NO: 18. In various embodiments, the CPP does not have the sequence of SEQ ID NO: 16.

In various embodiments, the CPP can have an N-terminal glycine added. For example, TATκ28 and TAT28 would otherwise have an N-terminal aspartate residue, which has a low stability. Adding an N-terminal glycine to the sequence can increase protein stability via the N-end rule. Accordingly, in some embodiments, any of the fusion proteins that have a leader signal polypeptide can have a glycine added at the C-terminal end of the leader signal polypeptide, such that upon cleavage of the leader signal polypeptide, the new N-terminus of the fusion protein will begin with glycine. In an analogous manner, those fusion proteins lacking a leader signal polypeptide can also have a glycine added between the N-terminal methionine and the remainder of the fusion protein. Also in analogous manner, those fusion proteins having a CPP other than TAT28 or TATκ28, can also have a glycine added between a leader signal polypeptide and a CPP.

### Fusion Proteins Comprising CDKL5 Variants

As described above, CDKL5 variants can be used in fusion proteins, such as proteins that also contain a CPP. Other polypeptides can also be incorporated into such fusion proteins, such as leader signal polypeptides to enhance protein secretion or tags for detecting and/or purifying the fusion proteins, as well as linker polypeptides that can be used to link functional polypeptides.

Examples of leader signal polypeptides include, but are not limited to, modified fragments of human immunoglobulin heavy chain binding protein (modified BiP, e.g. SEQ ID NO: 48, SEQ ID NO: 51, SEQ ID NO: 52 or SEQ ID NO: 53) or murine IgK chain leader polypeptide (SEQ ID NO: 49, e.g. pSecTag2 from ThermoFisher vectors). Examples of modified BiP signal polypeptides include those described in U.S. Pat. No. 9,279,007, which is hereby incorporated by reference in its entirety.

Examples of tags that can be added to the fusion proteins include, but are not limited to, epitope tags (e.g. MYC, HA, V5, NE), glutathione S-transferase (GST), maltose-binding protein (MBP), calmodulin-binding peptide (CBP), FLAG®, 3xFLAG® and polyhistidine.

### Formulations, Methods of Treatment and Use

The recombinant protein (e.g., CDKL5 variant or fusion protein), can be formulated in accordance with the routine procedures as a pharmaceutical composition adapted for administration to human beings. For example, in one or more embodiments, a composition for intravenous administration is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

Recombinant protein (e.g., CDKL5 variant or fusion protein) (or a composition or medicament containing recombinant protein) is administered by an appropriate route. In one or more embodiments, the recombinant protein is administered intravenously. In other embodiments, recombinant protein is administered by direct administration to a target tissue, such as to heart or skeletal muscle (e.g., intramuscular; intraventricularly), or nervous system (e.g., direct injection into the brain; intrathecally). More than one route can be used concurrently, if desired.

The recombinant protein (e.g., CDKL5 variant or fusion protein) (or a composition or medicament containing recombinant protein) is administered in a therapeutically effective amount (e.g., a dosage amount that, when administered at regular intervals, is sufficient to treat the disease, such as by ameliorating symptoms associated with the disease, preventing or delaying the onset of the disease, and/or lessening the severity or frequency of symptoms of the disease). The amount which will be therapeutically effective in the treatment of the disease will depend on the nature and extent of the disease's effects. In addition, in vitro or in vivo assays may optionally be employed to help identify optimal dosage ranges. The precise dose to be employed will also depend on the route of administration, and the seriousness of the disease, and should be decided according to the judgment of a practitioner and each patient's circumstances. Effective doses may be extrapolated from dose-response curves derived from in vitro or animal model test systems.

The therapeutically effective amount of recombinant protein (e.g., CDKL5 variant or fusion protein) (or a composition or medicament containing recombinant protein) can be administered at regular intervals, depending on the nature and extent of the disease's effects, and/or on an ongoing basis. Administration at a “regular interval,” as used herein, indicates that the therapeutically effective amount is administered periodically (as distinguished from a one-time dose). The administration interval for a single individual need not be a fixed interval, but can be varied over time, depending on the needs of the individual.

The recombinant protein (e.g., CDKL5 variant or fusion protein) may be prepared for later use, such as in a unit dose vial or syringe, or in a bottle or bag for intravenous administration. Kits containing the recombinant protein (e.g., CDKL5 variant or fusion protein), as well as optional excipients or other active ingredients, such as other drugs, may be enclosed in packaging material and accompanied by instructions for reconstitution, dilution or dosing for treating a subject in need of treatment, such as a patient having a CDKL5 deficiency, Rett syndrome, or a Rett syndrome variant.

### Methods of Production

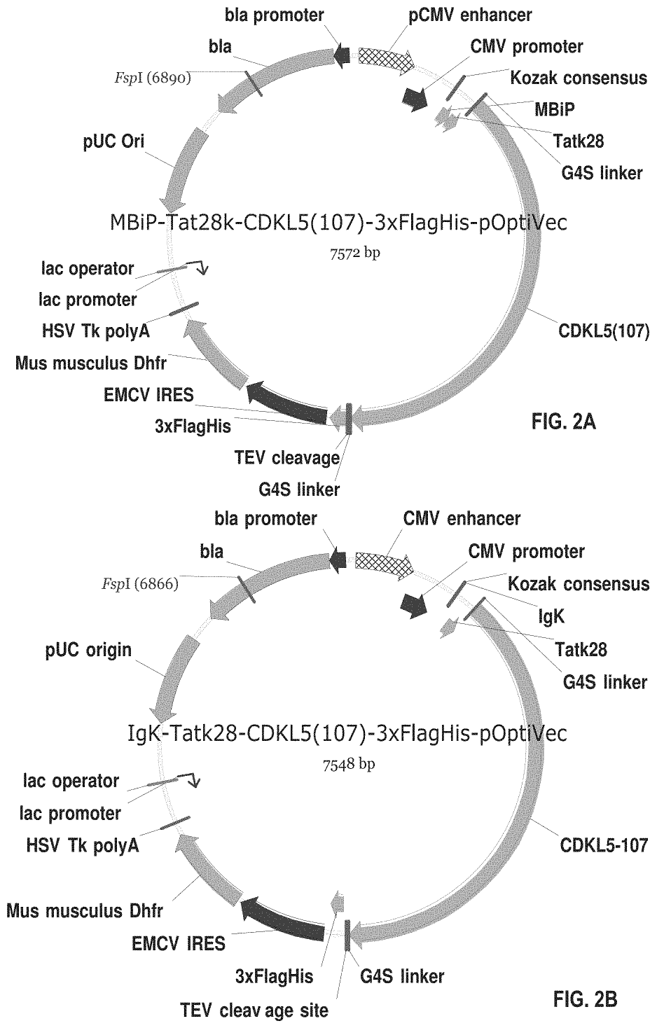

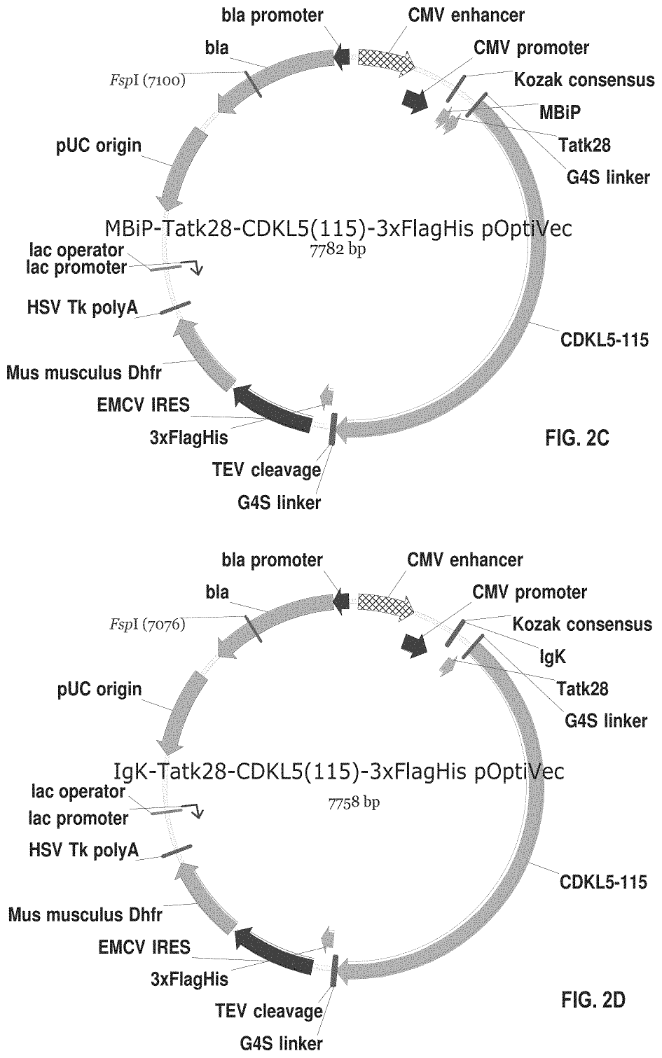

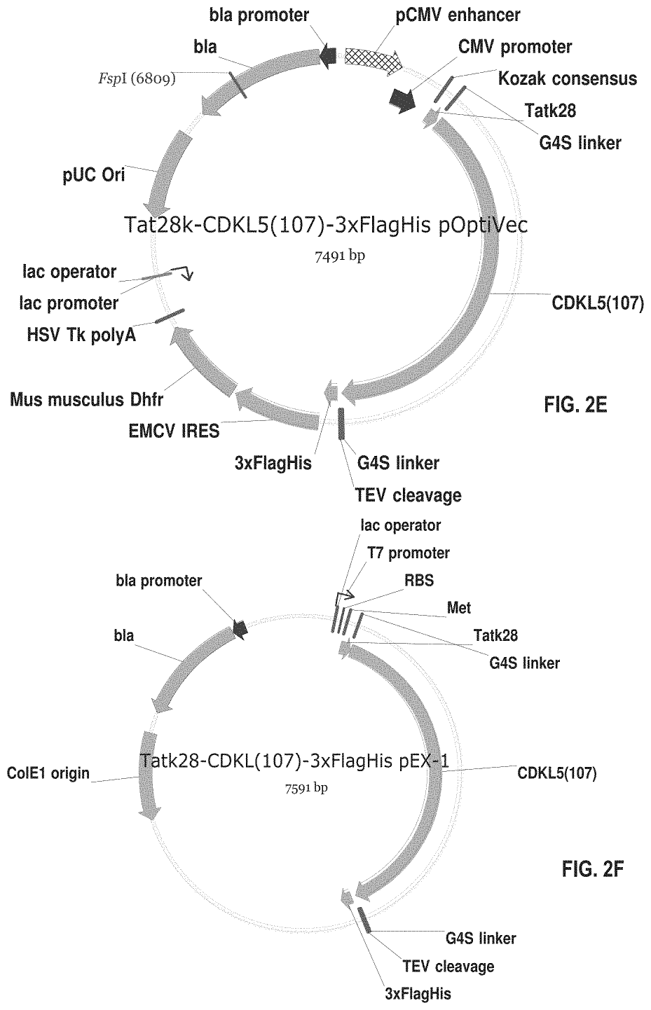

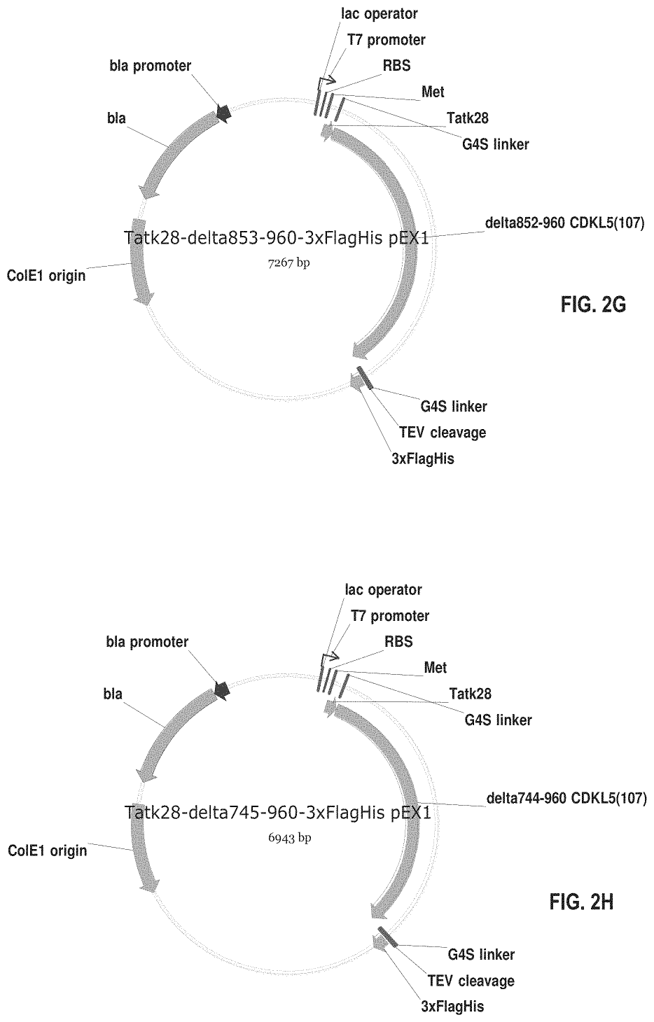

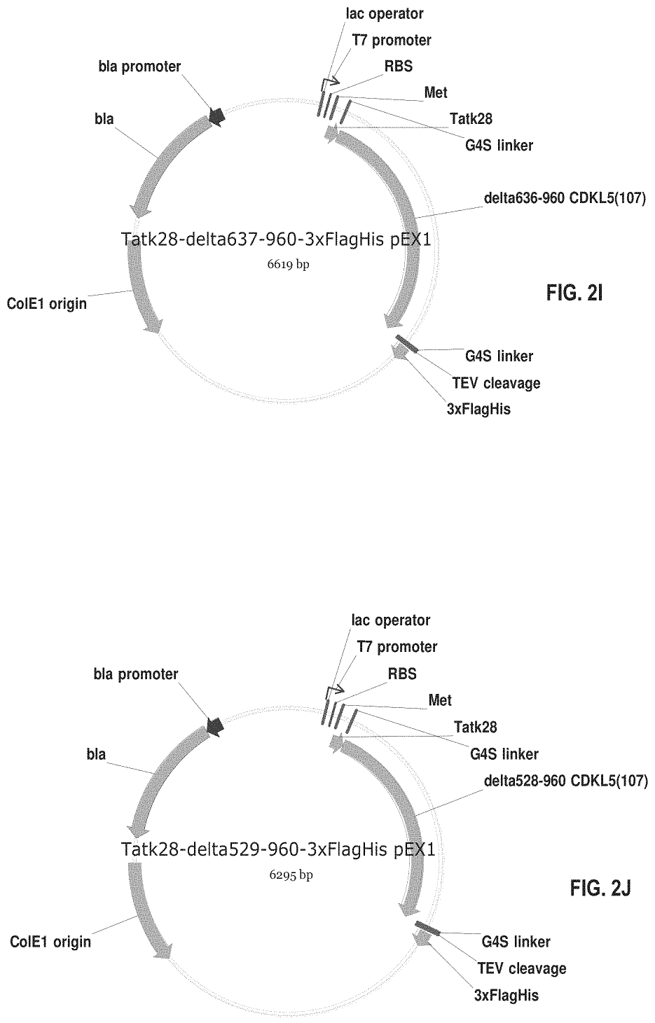

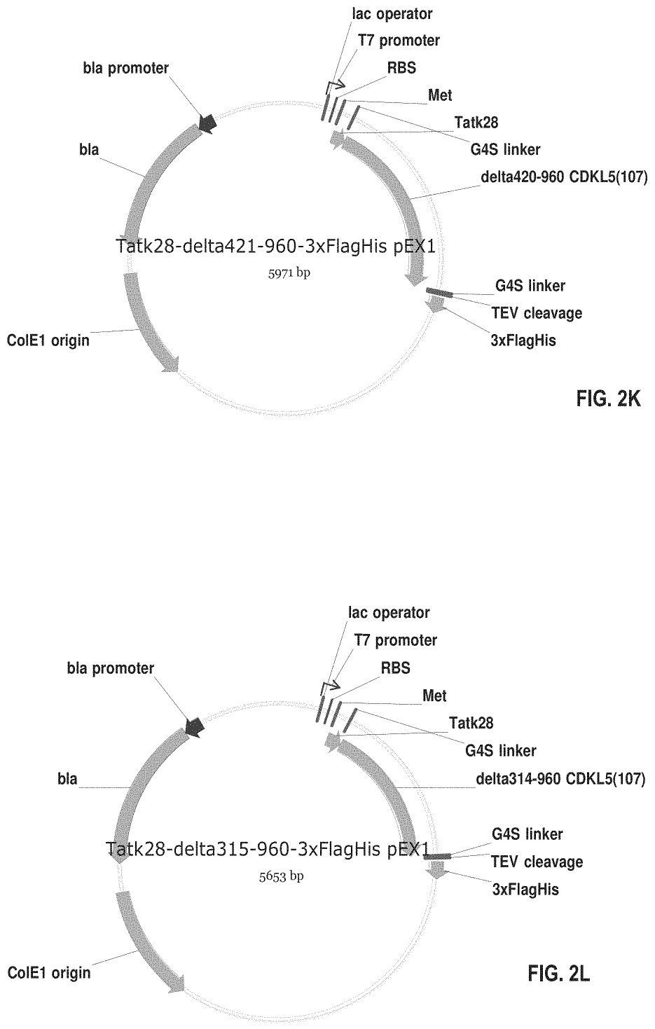

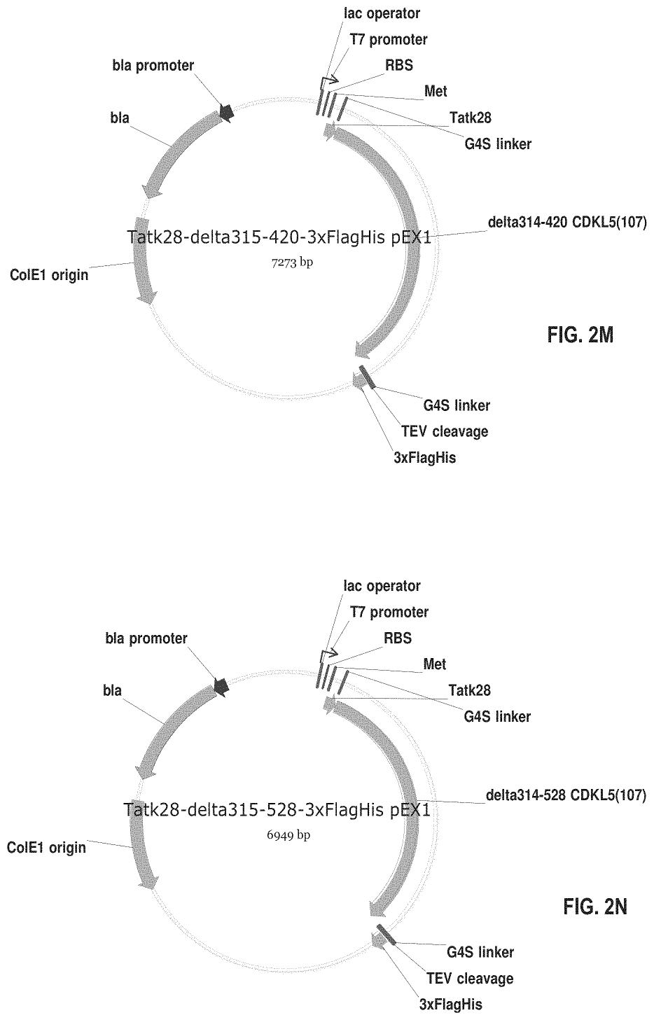

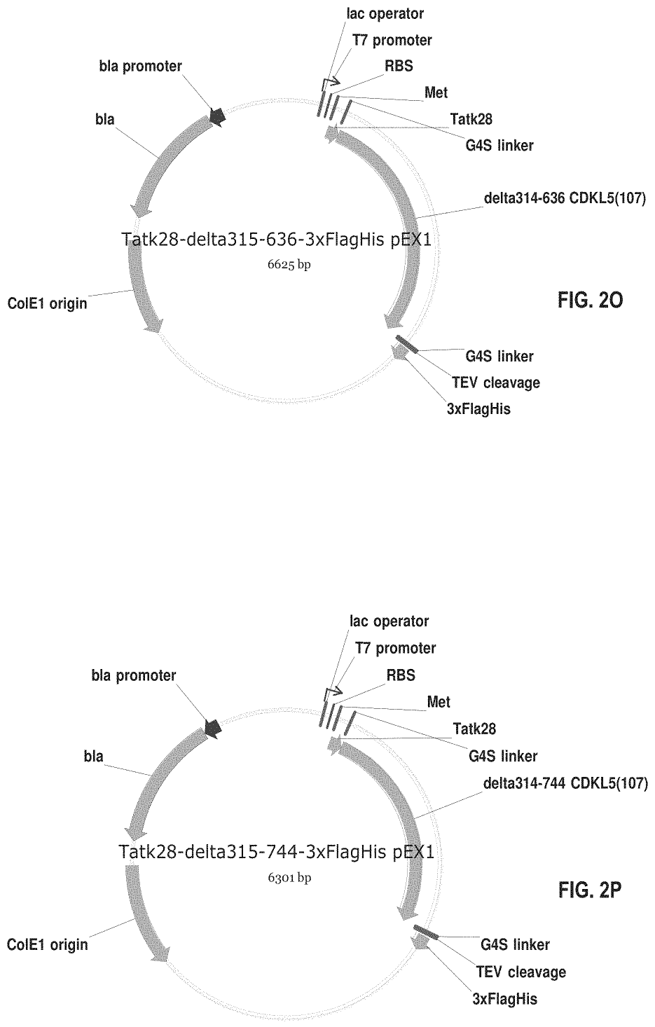

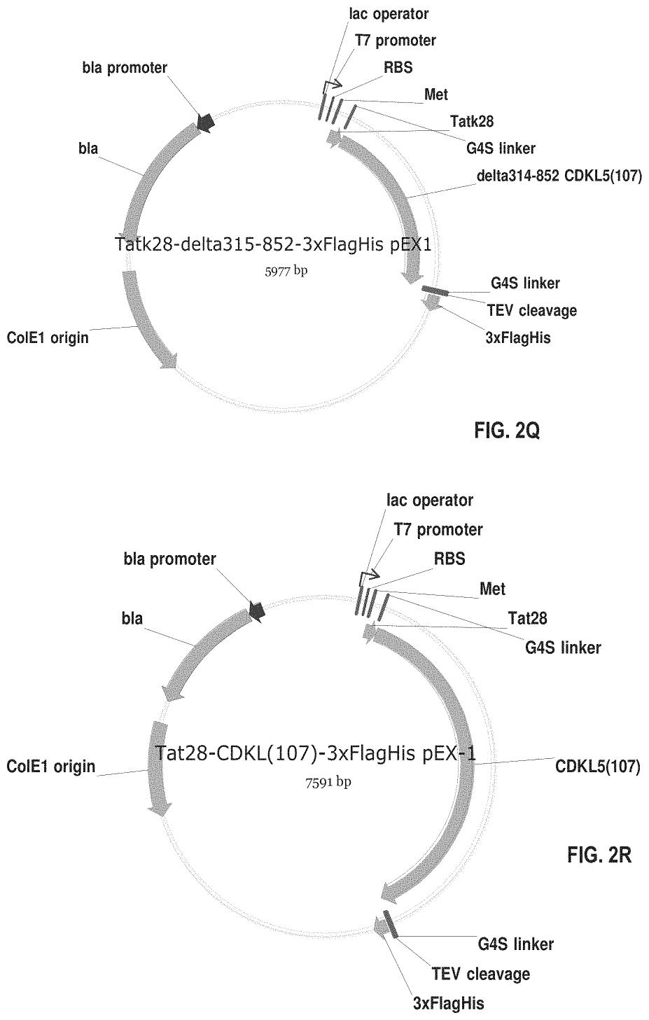

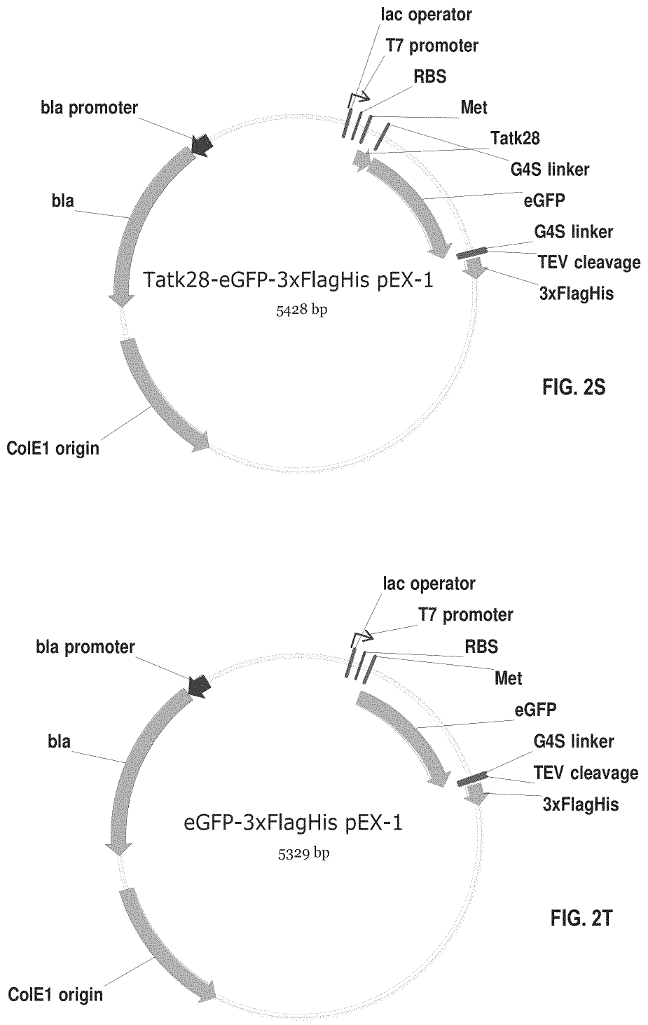

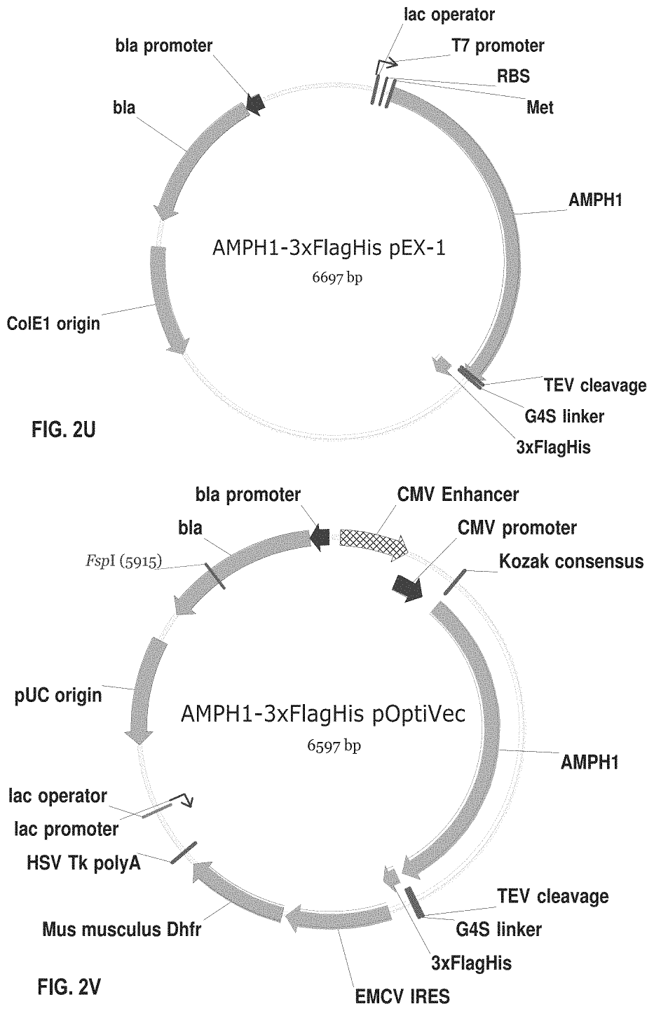

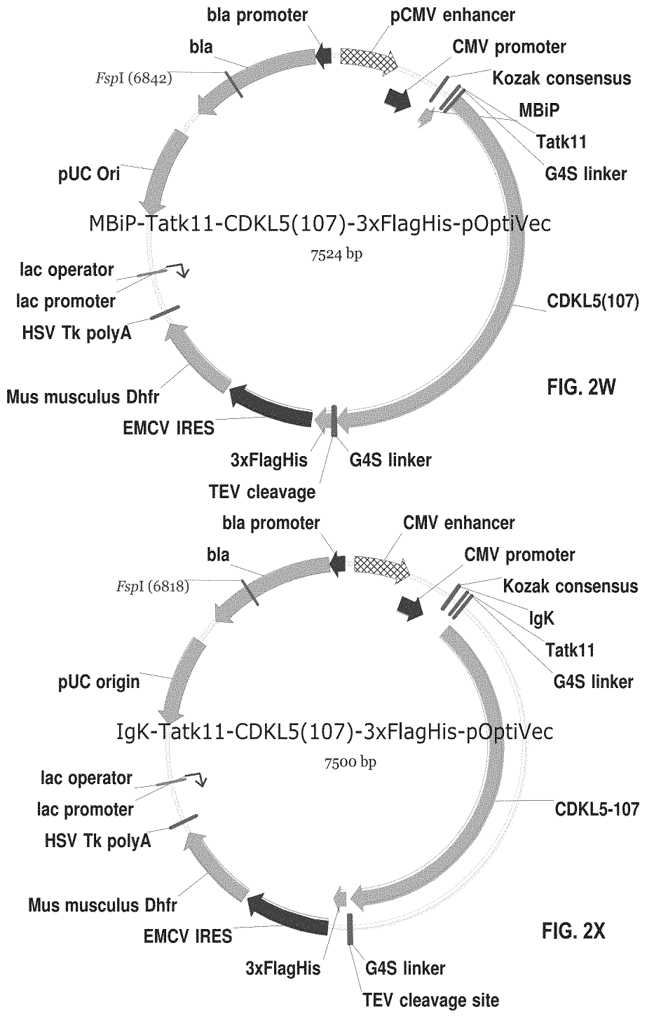

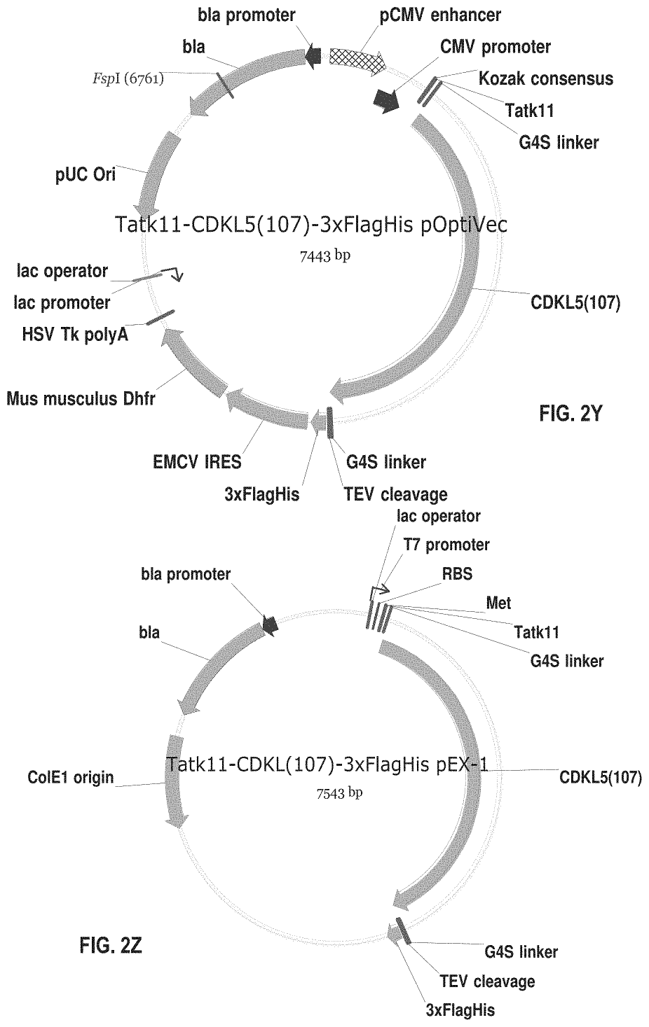

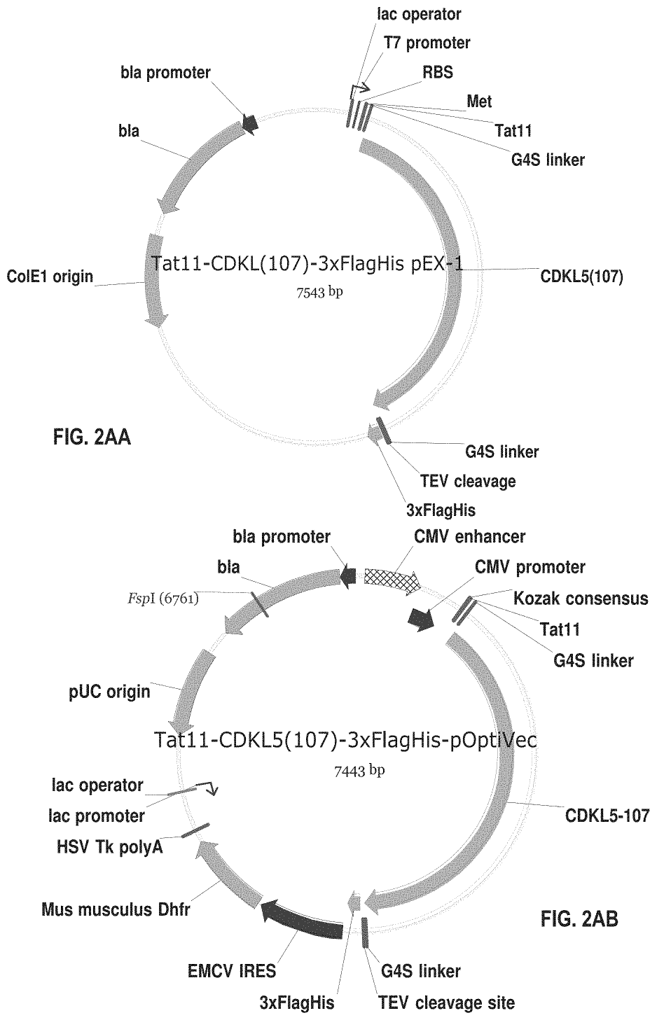

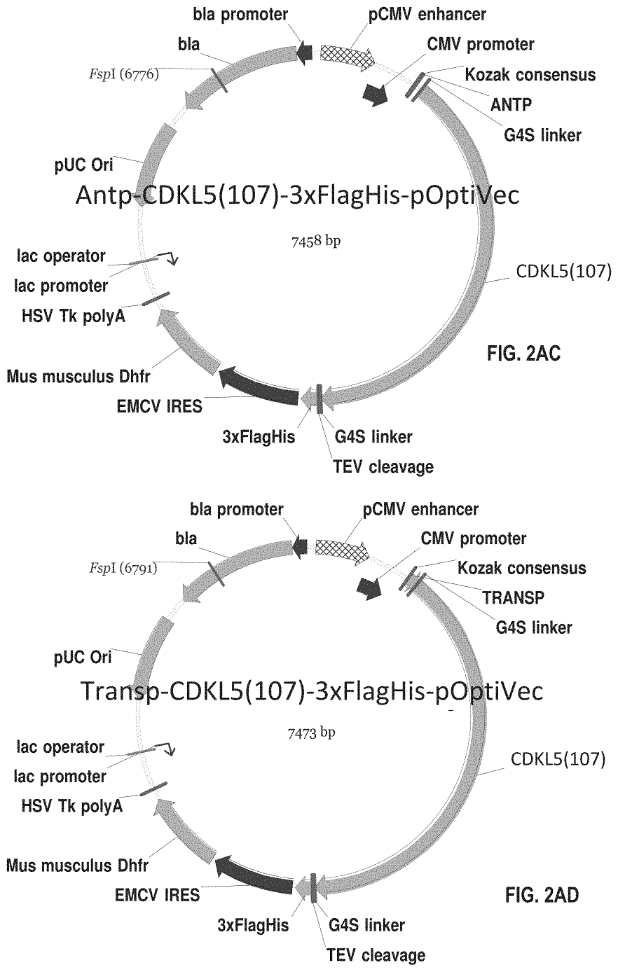

The recombinant protein (e.g. CDKL5 variant or fusion protein) can be expressed in and secreted from host cells using appropriate vectors. For example, mammalian cells (e.g., CHO, HeLa or HEK cells) or bacterial cells (e.g., E. coli or P. haloplanktis TAC 125 cells) can be used. Exemplary plasmids are described in the examples below and shown in FIGS. 2A-2AD. Those of skill in the art can select alternative vectors suitable for transforming, transfecting, or transducing cells to produce the CDKL5 variants and fusion proteins described herein.

After expression and secretion, recombinant protein can be recovered and purified from the surrounding cell culture media using standard techniques. Alternatively, recombinant protein can be isolated and purified directly from cells, rather than the medium.

### EXAMPLES

### Example 1—CDKL5 Fusion Proteins

FIGS. 2A-2AD show plasmids for expressing fusion proteins in suitable cells, such as mammalian cells (e.g., CHO cells) or bacterial cells (e.g., E. coli cells). These proteins have the amino acid sequences set forth in SEQ ID NOS: 19-46. The numbering of the deletions or truncations is relative to the full-length CDKL5₁₀₇ polypeptide (1-960). In those constructs wherein CDKL5 is fused C-terminally to additional N-terminal amino acid sequences, the initial methionine (amino acid 1) of CDKL5 is removed. In these constructs, the CDKL5 polypeptide begins with the second amino acid, lysine. The abbreviations used in FIGS. 2A-2AD and SEQ ID NOS: 19-46 and 54-55 are summarized in Table 1 below:

TABLE 1

| Features | Description |
|---|---|
| pOptiVec | expression vector for CHO DG44 cells, using pCMV promoter for high |
| | expression of recombinant protein; from ThermoFisher Scientific Inc. |
| pEX-1 | expression vector for bacterial cells, using T7 promoter for high |
| | expression of recombinant protein; from OriGene Technologies, Inc |
| pCMV | allows high expression level of recombinant protein |
| enhancer and | |
| promoter | |
| Kozak | for proper initiation of translation |
| consensus | |
| MBiP | modified BiP leader signal polypeptide (from U.S. Pat. No. 9,279,007; |
| | SEQ ID NO. 20) for secretion of recombinant protein; |
| | MKLSLVAAMLLLLSLVAAMLLLLSAARA |
| Igκ | murine Igκ chain leader polypeptide for secretion of recombinant |
| | protein (from ThermoFisher vectors; eg. pSecTag2); |
| | METDTLLLWVLLLWVPGSTG |
| Tatk28, | refers to the TATκ28 peptide, |
| Tatk28p, | GDAAQPARRARRTKLAAYARKAARQARA |
| Tk28p | |
| Tat28, | refers to the TAT28 peptide, |
| Tat28p, | GDAAQPARRARRTKLAAYGRKKRRQRRR |
| Tt28p | |
| Antp | Antennapedia peptide, |
| | RQIKIWFQNRRMKWKK |
| Transp | Transportan peptide, |
| | RQIKIWFQNRRMKWKK |
| G4S linker | a short linker consisting of 4 glycine and 1 serine |
| CDKL5(107) | human CDKL5-107 isoform |
| CDKL5(115) | human CDKL5-115 isoform |
| delta###-### | delta###-### refers to the deletion of amino acids to form truncated |
| | forms of protein |
| AMPH1 | gene encoding human Amphiphysin1 |
| eGFP | gene encoding the enhanced Green Fluorescent Protein; allows for |
| | detection using anti-GFP or fluorescence |
| TEV | TEV protease cleavage recognition site; allows removal of 3XFLAG-HIS |
| cleavage | tag (or other tags) after initial purification |
| 3XFlagHis | 3XFLAG tag, followed by Glycine-Alanine-Proline (a short linker), |
| | and 6xHis tag; Flag and His tag allows detection of fusion protein |
| | with anti-Flag and anti-His and allows purification |
| EMCV IRES | Internal Ribosome Entry Site from the Encephalomyocarditis Virus |
| | allows for cap-independent translation of DHFR |
| DHFR | Mus musculus (mouse) DHFR allows auxotrophic selection of transfected |
| | DG44 cells and for genomic amplification of stable cell lines using |
| | methothrexate (Mtx) |
| HSV Tk | Herpes Simplex Virus Thymidine Kinase polyadenylation signal allows |
| polyA | for efficient transcription termination and polyadenylation of mRNA |
| pUC Ori | pUC origin allows for high-copy number replication and growth in |
| | E. coli cells |
| bla promoter | promoter for ampicillin (bla) resistance gene |
| bla | ampicillin resistance gene (β-lactamase) |

FIG. 2A shows an exemplary plasmid for expressing the fusion protein of SEQ ID NO: 19 in CHO cells. This fusion protein comprises the modified BiP leader signal polypeptide, TATκ28 and the full-length human CDKL5₁₀₇ isoform.

FIG. 2B shows an exemplary plasmid for expressing the fusion protein of SEQ ID NO: 20 in CHO cells. This fusion protein comprises the murine Igκ chain leader polypeptide, TATκ28 and the full-length human CDKL5₁₀₇ isoform.

FIG. 2C shows an exemplary plasmid for expressing the fusion protein of SEQ ID NO: 21 in CHO cells. This fusion protein comprises the modified BiP leader signal polypeptide, TATκ28 and the full-length human CDKL5₁₁₅ isoform.

FIG. 2D shows an exemplary plasmid for expressing the fusion protein of SEQ ID NO: 22 in CHO cells. This fusion protein comprises the murine Igκ chain leader polypeptide, TATκ28 and the full-length human CDKL5₁₁₅ isoform.

FIG. 2E shows an exemplary plasmid for expressing the fusion protein of SEQ ID NO: 23 in CHO cells. This fusion protein comprises TATκ28 and the full-length human CDKL5₁₀₇ isoform.

FIG. 2F shows an exemplary plasmid for expressing the fusion protein of SEQ ID NO: 24 in E. coli cells. This fusion protein comprises TATκ28 and the full-length human CDKL5₁₀₇ isoform.

FIG. 2G shows an exemplary plasmid for expressing the fusion protein of SEQ ID NO: 25 in E. coli cells. This fusion protein comprises TATκ28 and the CDKL5₁₀₇ variant of Construct 2.

FIG. 2H shows an exemplary plasmid for expressing the fusion protein of SEQ ID NO: 26 in E. coli cells. This fusion protein comprises TATκ28 and the CDKL5₁₀₇ variant of Construct 3.

FIG. 21 shows an exemplary plasmid for expressing the fusion protein of SEQ ID NO: 27 in E. coli cells. This fusion protein comprises TATκ28 and the CDKL5₁₀₇ variant of Construct 4.

FIG. 2J shows an exemplary plasmid for expressing the fusion protein of SEQ ID NO: 28 in E. coli cells. This fusion protein comprises TATκ28 and the CDKL5₁₀₇ variant of Construct 5.

FIG. 2K shows an exemplary plasmid for expressing the fusion protein of SEQ ID NO: 29 in E. coli cells. This fusion protein comprises TATκ28 and the CDKL5₁₀₇ variant of Construct 6.

FIG. 2L shows an exemplary plasmid for expressing the fusion protein of SEQ ID NO: 30 in E. coli cells. This fusion protein comprises TATκ28 and the CDKL5₁₀₇ variant of Construct 7.

FIG. 2M shows an exemplary plasmid for expressing the fusion protein of SEQ ID NO: 31 in E. coli cells. This fusion protein comprises TATκ28 and the CDKL5₁₀₇ variant of Construct 8.

FIG. 2N shows an exemplary plasmid for expressing the fusion protein of SEQ ID NO: 32 in E. coli cells. This fusion protein comprises TATκ28 and the CDKL5₁₀₇ variant of Construct 9.

FIG. 20 shows an exemplary plasmid for expressing the fusion protein of SEQ ID NO: 33 in E. coli cells. This fusion protein comprises TATκ28 and the CDKL5₁₀₇ variant of Construct 10.

FIG. 2P shows an exemplary plasmid for expressing the fusion protein of SEQ ID NO: 34 in E. coli cells. This fusion protein comprises TATκ28 and the CDKL5₁₀₇ variant of Construct 11.

FIG. 2Q shows an exemplary plasmid for expressing the fusion protein of SEQ ID NO: 35 in E. coli cells. This fusion protein comprises TATκ28 and the CDKL5₁₀₇ variant of Construct 12.

FIG. 2R shows an exemplary plasmid for expressing the fusion protein of SEQ ID NO: 36 in E. coli cells. This fusion protein comprises TAT28 and the full-length human CDKL5₁₀₇ isoform.

FIG. 2S shows an exemplary plasmid for expressing the fusion protein of SEQ ID NO: 37 in E. coli cells. This fusion protein comprises TATκ28 and enhanced Green Fluorescent Protein (eGFP).

FIG. 2T shows an exemplary plasmid for expressing the fusion protein of SEQ ID NO: 38 in E. coli cells. This fusion protein comprises eGFP without a CPP.

FIG. 2U shows an exemplary plasmid for expressing the fusion protein of SEQ ID NO: 39 in E. coli cells. This fusion protein comprises human Amphiphysinl (AMPH1).

FIG. 2V shows an exemplary plasmid for expressing the fusion protein of SEQ ID NO: 40 in CHO cells. This fusion protein comprises human Amphiphysinl (AMPH1).

FIG. 2W shows an exemplary plasmid for expressing the fusion protein of SEQ ID NO: 41 in CHO cells. This fusion protein comprises the modified BiP leader signal polypeptide, TATκ11 and the full-length human CDKL5₁₀₇ isoform.

FIG. 2X shows an exemplary plasmid for expressing the fusion protein of SEQ ID NO: 42 in CHO cells. This fusion protein comprises the murine Igκ chain leader polypeptide, TATκ11 and the full-length human CDKL5₁₀₇ isoform.

FIG. 2Y shows an exemplary plasmid for expressing the fusion protein of SEQ ID NO: 43 in CHO cells. This fusion protein comprises TATκ11 and the full-length human CDKL5₁₀₇ isoform without a leader signal polypeptide.

FIG. 2Z shows an exemplary plasmid for expressing the fusion protein of SEQ ID NO: 44 in E. coli cells. This fusion protein comprises TATκ11 and the full-length human CDKL5₁₀₇ isoform without a leader signal polypeptide.

FIG. 2AA shows an exemplary plasmid for expressing the fusion protein of SEQ ID NO: 45 in E. coli cells. This fusion protein comprises TAT11 and the full-length human CDKL5₁₀₇ isoform without a leader signal polypeptide.

FIG. 2AB shows an exemplary plasmid for expressing the fusion protein of SEQ ID NO: 46 in CHO cells. This fusion protein comprises TAT11 and the full-length human CDKL5₁₀₇ isoform without a leader signal polypeptide.

FIG. 2AC shows an exemplary plasmid for expressing the fusion protein of SEQ ID NO: 54 in CHO cells. This fusion protein comprises the Antennapedia CPP and the full-length human CDKL5₁₀₇ isoform without a leader signal polypeptide.

FIG. 2AD shows an exemplary plasmid for expressing the fusion protein of SEQ ID NO: 55 in CHO cells. This fusion protein comprises the Transportan CPP and the full-length human CDKL5₁₀₇ isoform without a leader signal polypeptide.

The CDKL5 fusion proteins of SEQ ID NOS: 19-36 and 41-46 will be expressed and evaluated for activity using the plasmids of FIGS. 2A-2R and 2W-2AB, respectively. Human amphiphysin 1 (AMPH1) will be the substrate in the CDKL5 kinase assays. The plasmids of FIGS. 2U and 2V will be used to express affinity-tagged AMPH1 (SEQ ID NOS: 39 and 40) for the CDKL5 kinase assays. Affinity-tagged eGFP alone (SEQ ID NO. 38) as well as affinity-tagged TATκ28-eGFP (SEQ ID NO. 37) will serve as controls for the CDKL5 fusion proteins, which will be expressed using the plasmids of FIGS. 2S and 2T, respectively.

Various CDKL5 fusion proteins were expressed in CHO and HEK cells, as well as using in vitro transcription/translation with HeLa cell lysates. Briefly, CHO-S cells (20×\hat{ }6 cells) were electroporated using Maxcyte STX with 8 plasmids: (1) pOptiVec empty vector; 2) TATk28-CDKL5-107-3xFlagHis; 3) TATk11-CDKL5-107-3xFlagHis; 4) TAT11-CDKL5-107-3xFlagHis; 5) TAT28-CDKL5-107-3xFlagHis; 6) ANTP-CDKL5-107-3xFlagHis; 7) TRANSP-CDKL5-107-3xFlagHis and 8) MBiP-TATK28-CDKL5-107-3xFlagHis (coding sequences being CHO codon-optimized). Cells were recovered in culture medium, and cultured for one day. Cells were harvested and lysed. For each transfection, 20 μg lysate was subjected to 4-12% BisTris SDS-PAGE, and transferred to nitrocellulose blot using the iBlot2 system. The blot was blocked in 5% milk in 1xTBS-T. Blot was subjected to Western blot by incubating with 1:2000 dilution of rabbit anti-His antibody overnight. After a series of washes, blot was incubated with 1:10000 anti-rabbit IgG DyaLight 680 secondary antibody. Additional washes were performed. Blot was imaged on Licor Odyssey scanner. Blot confirmed expression of the CDKL5 fusion proteins.

HEK293F cells (8×10\hat{ }6 cells) were transfected with FuGeneHD (24 μl FuGeneHD: 8 μg DNA ratio) and 7 plasmids: 1) empty pOptiVec; 2) TATk11-CDKL5_107-3xFlagHis; 3) TAT11-CDKL5_1-FH; 4) TAT28-CDKL5_1-FH; 5) ANTP-CDKL5_107-3xFlagHis; TRANSP-CDKL5_107-3xFlagHis and 7) TATk28-CDKL5_107-3xFlagHis (coding sequences being human codon-optimized). Cells were incubated and harvested 2 days post transfection. Cells were lysed, and 20 μg lysate was subjected to 4-12% BisTris SDS-PAGE, and transferred to nitrocellulose blot using the iBlot2 system. The blot was blocked in 5% milk in 1xTBS-T. Blot was subjected to Western blot by incubating with 1:2000 dilution of rabbit anti-His antibody overnight. After a series of washes, blot was incubated with 1:10000 anti-rabbit IgG DyaLight 680 secondary antibody. Additional washes were performed. Blot was imaged on Licor Odyssey scanner. Blot confirmed expression of the CDKL5 fusion proteins.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

1-23. (canceled)

24. A fusion protein comprising a CDKL5 polypeptide and a cell-penetrating polypeptide operatively coupled together, wherein the CDKL5 polypeptide comprises a sequence having at least 98% sequence identity to SEQ ID NO: 1 or SEQ ID NO: 47 and the cell-penetrating polypeptide comprises a sequence having at least 90% sequence identity to SEQ ID NO: 13, SEQ ID NO: 14, SEQ ID NO: 15, SEQ ID NO: 17, SEQ ID NO: 18 or SEQ ID NO: 50.

25. The fusion protein of claim 24, wherein the cell-penetrating polypeptide comprises a sequence having at least 95% sequence identity to SEQ ID NO: 13, SEQ ID NO: 14, SEQ ID NO: 15, SEQ ID NO: 17, SEQ ID NO: 18 or SEQ ID NO: 50.

26. The fusion protein of claim 25, wherein the cell-penetrating polypeptide comprises a sequence having 100% sequence identity to SEQ ID NO: 13, SEQ ID NO: 14, SEQ ID NO: 15, SEQ ID NO: 17, SEQ ID NO: 18 or SEQ ID NO: 50.

27. The fusion protein of claim 24, further comprising a leader signal polypeptide.

28. The fusion protein of claim 27, wherein the leader signal polypeptide comprises a sequence having at least 90% sequence identity to SEQ ID NO: 48, SEQ ID NO: 49, SEQ ID NO: 51, SEQ ID NO: 52 or SEQ ID NO: 53.

29. The fusion protein of claim 28, wherein the leader signal polypeptide comprises a sequence having 100% sequence identity to SEQ ID NO: 48, SEQ ID NO: 49, SEQ ID NO: 51, SEQ ID NO: 52 or SEQ ID NO: 53.

30. A fusion protein comprising a CDKL5 polypeptide and a leader signal polypeptide operatively coupled together, wherein the CDKL5 polypeptide comprises a sequence having at least 98% sequence identity to SEQ ID NO: 1 or SEQ ID NO: 47 and the leader signal polypeptide comprises a sequence having at least 90% sequence identity to SEQ ID NO: 48, SEQ ID NO: 51, SEQ ID NO: 52 or SEQ ID NO: 53.

31. The fusion protein of claim 30, wherein the leader signal polypeptide comprises a sequence having 100% sequence identity to SEQ ID NO: 48, SEQ ID NO: 51, SEQ ID NO: 52 or SEQ ID NO: 53.

32. The fusion protein of claim 30 or 31, further comprising a cell-penetrating polypeptide.

33. The fusion protein of claim 32, wherein the cell-penetrating polypeptide comprises a sequence having at least 90% sequence identity to SEQ ID NO: 13, SEQ ID NO: 14, SEQ ID NO: 15, SEQ ID NO: 16, SEQ ID NO: 17, SEQ ID NO: 18 or SEQ ID NO: 50.

34. The fusion protein of claim 33, wherein the cell-penetrating polypeptide comprises a sequence having at least 95% sequence identity to SEQ ID NO: 13, SEQ ID NO: 14, SEQ ID NO: 15, SEQ ID NO: 16, SEQ ID NO: 17, SEQ ID NO: 18 or SEQ ID NO: 50.

35. The fusion protein of claim 34, wherein the cell-penetrating polypeptide comprises a sequence having100% sequence identity to SEQ ID NO: 13, SEQ ID NO: 14, SEQ ID NO: 15, SEQ ID NO: 16, SEQ ID NO: 17, SEQ ID NO: 18 or SEQ ID NO: 50.

36. A pharmaceutical formulation comprising the fusion claim 24; and a pharmaceutically acceptable carrier.

37. A method of treating a CDKL5-mediated neurological disorder, the method comprising administering the formulation of claim 36 to a patient in need thereof.

38. The method of claim 37, wherein the formulation is administered intrathecally, intravenously, intracisternally, intracerebroventrically or intraparenchymally.

39. The method of claim 37, wherein the formulation is administered intrathecally or intravenously.

40. The method of claim 37, wherein the CDKL5-mediated neurological disorder is one or more of a CDKL5 deficiency or an atypical Rett syndrome caused by a CDKL5 mutation or deficiency.

41. A method of producing the fusion protein of claim 24, the method comprising:
expressing the fusion protein; and
purifying the fusion protein.

42. The method of claim 41, wherein the fusion protein is expressed in Chinese hamster ovary (CHO) cells, HeLa cells, human embryonic kidney (HEK) cells or Escherichia coli cells.

43. A polynucleotide encoding the fusion protein of claim 24.

44. A vector comprising the polynucleotide of claim 43.
